(19) United States
(12) Patent Application Publication
(10) Pub. No.: US 2018/0207108 A1
(43) Pub. Date: Jul. 26, 2018

(54) Method for Manufacturing Asenapine-Containing Patch

(71) Applicant: HISAMITSU PHARMACEUTICAL CO., INC., Tosu-shi, Saga (JP)

(72) Inventors: Atsushi SONOBE, Tsukuba-shi, Ibaraki (JP); Takashi YASUKOCHI, Tsukuba-shi, Ibaraki (JP)

(21) Appl. No.: 15/744,956

(22) PCT Filed: Jul. 21, 2016
(86) PCT No.: PCT/JP2016/071449

§ 371 Date: Jan. 15, 2018

(30) Foreign Application Priority Data
Jul. 27, 2015 (JP) 2015-147516

(51) Int. Cl. A61K 9/70; A61K 31/407; A61K 47/02; A61K 47/32

(52) U.S. Cl. CPC A61K 9/7053; A61K 47/32; A61K 47/02; A61K 31/407

(57) ABSTRACT

The present invention provides a method for manufacturing a patch comprising a backing and an adhesive layer laminated on the backing, comprising: a step of mixing an acid addition salt of asenapine, a (meth)acrylic acid ester (co)polymer and a desalting agent to obtain an adhesive composition; and a step of shaping the adhesive composition to obtain the adhesive layer.

### TECHNICAL FIELD

The present invention relates to a method for manufacturing an asenapine-containing patch.

### BACKGROUND ART

Asenapine is a compound known as a therapeutic agent of central nervous system diseases, in particular, schizophrenia. A patch containing asenapine is described in, for example, Patent Literatures 1 to 4. A patch containing asenapine free base is known to be excellent in the skin permeability of asenapine.

### CITATION LIST

### Patent Literature

Patent Literature 1: WO 2010/127674

Patent Literature 2: WO 2014/017593

Patent Literature 3: WO 2014/017594

Patent Literature 4: WO 2014/017595

### SUMMARY OF INVENTION

### Technical Problem

Meanwhile, when the patch comprises an adhesive layer containing a (meth)acrylic acid ester (co)polymer, use of an asenapine free base in the manufacturing of the patch may lead to easy degradation of asenapine. Therefore, an object of the present invention is to provide a method for manufacturing the patch in which asenapine is not easily degraded.

### Solution to Problem

In processes of investigation of methods in which asenapine is not easily degraded in the manufacturing of the patch, the present inventors have found that degradation of asenapine is reduced when the adhesive layer comprises a metal maleate. They further found that the amounts of degradation products produced of asenapine can be more reduced when an adhesive composition comprising an acid addition salt of asenapine and a desalting agent is used in the manufacturing of the patch, compared to the case in which an adhesive composition comprising an asenapine free base and a metal maleate is used.

Meanwhile, according to the investigation of the present inventors, in the manufacturing of a patch comprising an adhesive layer containing a rubber adhesive, degradation of asenapine was hardly observed in the manufacturing of the patch. The present inventors suppose that degradation of asenapine in the manufacturing of the patch tends to be caused easily by contact with other components in the adhesive composition, heating or exposure to light such as UV rays.

Therefore, the present invention provides a method for manufacturing a patch comprising a backing and an adhesive layer laminated on the backing, comprising: a step of mixing an acid addition salt of asenapine, a (meth)acrylic acid ester (co)polymer and a desalting agent to obtain an adhesive composition; and a step of shaping the adhesive composition to obtain the adhesive layer.

It is preferable that the acid in the acid addition salt of asenapine be selected from the group consisting of hydrochloric acid, hydrobromic acid, hydriodic acid, phosphoric acid, acetic acid, propionic acid, glycolic acid, maleic acid, malonic acid, succinic acid, tartaric acid, citric acid, ascorbic acid, salicylic acid and benzoic acid. It is preferable that the desalting agent be selected from the group consisting of an alkali metal hydroxide, an alkali metal salt, an alkaline-earth metal hydroxide, an alkaline-earth metal salt and a low molecular weight amine.

The present invention also provides a method for stabilizing asenapine in an adhesive composition comprising a (meth)acrylic acid ester (co)polymer, comprising: a step of mixing an acid addition salt of asenapine, a (meth)acrylic acid ester (co)polymer and a desalting agent to obtain the adhesive composition.

The present invention provides a method for inhibiting the degradation of asenapine in a manufacturing process of a patch comprising an adhesive layer comprising asenapine, comprising: a step of mixing an acid addition salt of asenapine, a (meth)acrylic acid ester (co)polymer and a desalting agent to obtain an adhesive composition; and a step of shaping the adhesive composition to obtain the adhesive layer.

### Advantageous Effects of Invention

According to the method for manufacturing a patch related to the present invention, the amounts of degradation products produced of asenapine in the manufacturing of the patch can be reduced.

### BRIEF DESCRIPTION OF DRAWINGS

FIG. 1 is a graph showing the amounts of degradation products produced of asenapine.

FIG. 2 is a graph showing the amounts of degradation products produced of asenapine.

FIG. 3 is a graph showing the amounts of degradation products produced of asenapine.

### DESCRIPTION OF EMBODIMENTS

In the specification, the term “(meth)acrylic acid” refers to either one or both of acrylic acid and methacrylic acid, and similar expressions are defined in the same manner.

The present invention will be described in detail below with showing embodiments of the present invention.

One embodiment of the present invention is a patch comprising a backing and an adhesive layer laminated on the backing. The patch can be manufactured by a method comprising: a step of mixing an acid addition salt of asenapine, a (meth)acrylic acid ester (co)polymer and a desalting agent to obtain an adhesive composition; and a step of shaping the adhesive composition to obtain the adhesive layer.

The backing may be any backing which can maintain the form of the patch, in particular, the adhesive layer. Examples of the material of the backing include polyethylene, polypropylene, polybutadiene, an ethylene-vinyl chloride copolymer, polyvinyl chloride, polyamide such as nylon, polyester, a cellulose derivative, and a synthetic resin such as polyurethane. Examples of the shape of the backing include a film, a sheet, a sheet-like porous body, sheet-like foam, fabric such as woven fabric, knitted fabric and nonwoven fabric, and laminated products thereof. The thickness of the backing is not specifically limited, but it is preferable that the thickness of the backing be normally about 2 to 3000 μm.

The adhesive layer is formed from an adhesive composition obtained by mixing an acid addition salt of asenapine, a (meth)acrylic acid ester (co)polymer and a desalting agent. The adhesive layer preferably do not contain a rubber adhesive. Examples of the rubber adhesive include natural rubber, polyisobutylene, an alkyl vinyl ether (co)polymer, polyisoprene, polybutadiene, a styrene-butadiene copolymer, a styrene-isoprene copolymer, a styrene-isoprene-styrene block copolymer, and silicone rubber.

The thickness of the adhesive layer is not specifically limited and may be 30 to 300 μm. If the thickness of the adhesive layer is over 300 μm, the patch tends to easily fall off, for example when clothes are put on or off.

Asenapine is a compound which is also referred to as (3aRS,12bRS)-5-chloro-2-methyl-2,3,3a,12b-tetrahydro-1H-dibenzo[2,3:6,7]oxepino[4,5-c]pyrrole and is represented by the following chemical formula. Asenapine has a plurality of optical isomers and may be any optical isomer, and may be a mixture of optical isomers such as a racemate. The acid added to asenapine is not specifically limited as long as it is pharmaceutically acceptable. The acid addition salt of asenapine may be anhydride or hydrate.

Examples of the acid include hydrochloric acid, hydrobromic acid, hydroiodic acid, phosphoric acid, acetic acid, propionic acid, glycolic acid, maleic acid, malonic acid, succinic acid, tartaric acid, citric acid, ascorbic acid, salicylic acid and benzoic acid. The acid addition salt of asenapine may be anhydride or hydrate. For example, asenapine maleate is commercially available as a therapeutic agent of schizophrenia.

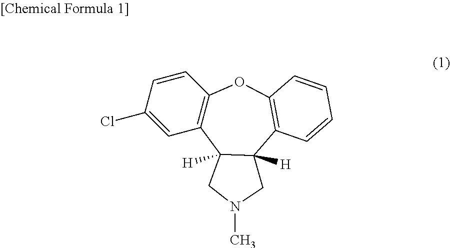 [SMILES: CN1C[C@H]2c3ccccc3Oc3ccc(Cl)cc3[C@@H]2C1]

The desalting agent may be any desalting agent which can convert an acid addition salt of asenapine into an asenapine free base by salt exchange reaction with an acid addition salt of asenapine. Namely, a desalting agent refers to a component which converts an acid addition salt of asenapine into an asenapine free base. Examples of the desalting agent include an alkali metal hydroxide, an alkali metal salt, an alkaline-earth metal hydroxide, an alkaline-earth metal salt and a low molecular weight amine. Examples of the alkali metal hydroxide include lithium hydroxide, sodium hydroxide and potassium hydroxide. Examples of the alkali metal salt include sodium carbonate, potassium carbonate, sodium bicarbonate, trisodium phosphate, disodium hydrogenphosphate, sodium dihydrogenphosphate, tripotassium phosphate, dipotassium hydrogenphosphate, potassium dihydrogenphosphate, sodium lactate, sodium citrate, disodium tartrate, sodium bitartrate and sodium oleate. The low molecular weight amine is an amine whose molecular weight is 30 to 300, and examples of the low molecular weight amine include monoethanolamine, diethanolamine, triethanolamine, isopropanolamine and diisopropanolamine. The desalting agent can be selected in consideration of pKa of the acid added to asenapine. When the desalting agent is sodium hydroxide, the degradation of agents is less in the manufacturing of the patch.

The (meth)acrylic acid ester (co)polymer is a component which gives adhesiveness to the adhesive layer, and examples of the (meth)acrylic acid ester (co)polymer include a (co)polymer of one or two or more types of (meth)acrylic acid alkyl ester. Examples of the (meth)acrylic acid alkyl ester include butyl (meth)acrylate, isobutyl (meth)acrylate, hexyl (meth)acrylate, octyl (meth)acrylate, 2-ethylhexyl (meth)acrylate and decyl (meth)acrylate.

The (meth)acrylic acid ester (co)polymer may be a copolymer formed from (meth)acrylic acid alkyl ester (a main monomer) and a comonomer. The comonomer may be any component which can copolymerize with (meth)acrylic acid alkyl ester. Examples of the comonomer include (meth)acrylic acid hydroxyalkyl ester, ethylene, propylene, styrene, vinyl acetate, N-vinylpyrrolidone and amide (meth)acrylate. One of these comonomers may be used alone or two or more may be used in combination.

Specific examples of the (meth)acrylic acid ester (co)polymer include DURO-TAK® 87-900A, DURO-TAK 87-2510, DURO-TAK 87-4287, DURO-TAK 87-2194 (trade name, manufactured by Henkel). DURO-TAK 87-2510 and DURO-TAK 87-4287 have a hydroxy group in its chemical structure and DURO-TAK 87-2194 has a carboxy group in its chemical structure.

The content of the (meth)acrylic acid ester (co)polymer is 50 to 98% by mass relative to the total mass of the adhesive layer and it is preferable that the content of the (meth)acrylic acid ester (co)polymer be 70 to 96% by mass. The content of the (meth)acrylic acid ester (co)polymer is calculated based on the mass of the solid content.

The adhesive layer may further contain other additives. Examples of the other additives include a tackifier resin, a plasticizer, an absorption promoting agent, a solubilizer, a stabilizing agent, a filler and a flavor.

The tackifier resin is a component which adjusts the adhesiveness of the adhesive layer. Examples of the tackifier resin include an alicyclic saturated hydrocarbon resin; rosin and rosin derivatives such as glycerine ester of rosin, hydrogenated rosin, glycerine ester of hydrogenated rosin, pentaerythritol ester of rosin and maleated rosin; a terpene tackifier resin; a petroleum tackifier resin. One of the tackifier resins may be used alone or two or more may be used in combination.

The absorption promoting agent is a component which adjust the skin permeability of asenapine or an acid addition salt of asenapine. The absorption promoting agent is not specifically limited as long as it is a compound whose absorption promoting action to skin is conventionally recognized, and examples of the absorption promoting agent include an aliphatic alcohol having 6 to 20 carbon atoms, an aliphatic ether having 6 to 20 carbon atoms, a fatty acid having 6 to 20 carbon atoms, a fatty ester having 6 to 20 carbon atoms, a fatty acid amide having 6 to 20 carbon atoms, glycerine, glycerine fatty esters, propylene glycols, propylene glycol fatty esters, polyethylene glycol and polyethylene glycol fatty esters, an aromatic organic acid, an aromatic alcohol, an aromatic organic acid ester, an aromatic organic ether (the above compounds may be saturated or unsaturated, and may be linear or branched, and may comprise a cyclic structure), lactic acid esters, acetic acid esters, a monoterpenes compound, a sesquiterpene compound, 1-dodecylazacycloheptane-2-one (Azone (trade name)) and derivatives thereof, sorbitan fatty esters (a series of Span®), a series of polysorbate (a series of Tween®), polyoxyethylene hydrogenated castor oils, polyoxyethylene alkylethers, sucrose fatty acid esters and a plant oil. Specific examples of the absorption promoting agent include caprylic acid, capric acid, caproic acid, lauric acid, myristic acid, palmitic acid, stearic acid, isostearic acid, oleic acid, linoleic acid, linolenic acid, lauryl alcohol, myristyl alcohol, oleyl alcohol, isostearyl alcohol, cetyl alcohol, methyl laurate, hexyl laurate, diethanolamide laurate, isopropyl myristate, myristyl myristate, octyldodecyl myristate, cetyl palmitate, isopropyl palmitate, salicylic acid, methyl salicylate, ethylene glycol salicylate, cinnamic acid, methyl cinnamate, cresol, cetyl lactate, lauryl lactate, ethyl acetate, propyl acetate, geraniol, thymol, eugenol, terpineol, 1-menthol, borneoroll, d-limonene, isoeugenol, isoborneol, nerol, dl-camphor, glycerin monocaprylate, glycerin monocaprate, glycerine monolaurate, glycerine monooleate, sorbitan monolaurate, sucrose monolaurate, polysorbate 20, propylene glycol, propylene glycol monolaurate, polyethylene glycol monolaurate, polyethylene glycol monostearate, polyoxyethylene lauryl ether, NIKKOL® HCO-60, Pyrrothiodecane®, olive oil and sorbitan monooleate. It is preferable that the absorption promoting agent be propylene glycol monolaurate, isopropyl palmitate, and it is more preferable that the absorption promoting agent be isopropyl palmitate, in that they remarkably improve the skin permeability of asenapine. One of the absorption promoting agents may be used alone or two or more may be used in combination.

When the adhesive layer contains the absorption promoting agent, it is preferable that the content of the absorption promoting agent be 2 to 40% by mass relative to the total mass of the adhesive layer. When the content of the absorption promoting agent is 2% by mass or more, the skin permeability of asenapine is increased and the asenapine-containing patch tends to exhibit enough pharmacological action. Further when the content of the absorption promoting agent is 40% by mass or less, skin irritancy tends not to be exhibited.

The solubilizer is a component which promotes the dissolution of asenapine and an acid addition salt thereof in the adhesive composition.

The stabilizing agent may be any stabilizing agent which can inhibit the production of free-radicals produced by the action of light such as UV rays, heat or active chemical species and the progression of the chain reaction of free-radicals. Inclusion of the stabilizing agent can further improve the stability of asenapine in the manufacturing of the patch. Examples of the stabilizing agent include tocopherol and ester derivatives thereof, ascorbic acid and ester derivatives thereof dibutylhydroxytoluene, butylhydroxyanisole, and 2-mercaptobenzimidazole. One of the stabilizing agents may be used alone or two or more may be used in combination. It is preferable that the stabilizing agent be dibutylhydroxytoluene in that physical properties (formability and appearance etc.) for a patch become appropriate and that asenapine is more stabilized.

When the adhesive layer contains the stabilizing agent, it is preferable that the content of the stabilizing agent be 0.1 to 3% by mass relative to the total mass of the adhesive layer. When the content of the stabilizing agent is 0.1 to 3% by mass, the stability of the components in the patch tends to be excellent.

The patch may further comprise a release liner. The release liner is laminated on the side opposite to the backing on the adhesive layer. When the patch comprises a release liner, the deposition of dirt etc. onto the adhesive layer tents to be reduced during storage.

The raw material of the release liner is not specifically limited, and liners generally known to those in the art may be used. Examples of the material of the release liner include polyester such as polyethylene terephthalate and polyethylene naphthalate; polyolefin such as polyethylene and polypropylene; films of polyvinyl chloride or polyvinylidene chloride etc.; laminated films of fine quality paper and polyolefin; films of Nylon (trade name) or aluminium etc. It is preferable that the material of the release liner be polypropylene or polyethylene terephthalate.

<Method of Manufacturing a Patch>

The patch of the present invention can be manufactured, for example, by the following method. First, an acid addition salt of asenapine, a (meth)acrylic acid ester (co)polymer and a desalting agent are mixed, and a solvent and other additives etc. are added there as needed and the mixture is mixed to obtain a homogeneous adhesive composition. Then, the obtained adhesive composition is applied onto one side of a backing in a given thickness and then is heated as needed to dry and remove the solvent, and the obtained sheet is cut into a desired size to obtain the patch. Heating conditions can be selected as appropriate depending on the solvent and it is preferable that temperature conditions be 60 to 120° C., and heating time is, for example, 2 to 30 minutes.

When a patch comprising a release liner is manufactured, an adhesive composition is applied to a backing, and then the solvent is dried and removed, and then the release liner can be laminated. Also, when a patch comprising a release liner is manufactured, the patch can be obtained by applying an adhesive composition onto one side of the release liner in a given thickness, and then heating the composition as needed to dry and remove the solvent, and layering a backing, and cutting the obtained sheet into a desired size.

The adhesive composition used in the above manufacturing methods contains an acid addition salt of asenapine, a (meth)acrylic acid ester (co)polymer and a desalting agent, and further contains a solvent and other additives etc. as needed.

The solvent may be a solvent which does not react with other components contained in the adhesive composition, and is added to adjust the viscosity of the adhesive composition. Examples of the solvent include toluene, ethanol, methanol and ethyl acetate. One of the solvent may be used alone or two or more may be used in combination. The content of the solvent may be adjusted in consideration of the viscosity of the adhesive composition.

It is preferable that the content of an acid addition salt of asenapine in the adhesive composition be 0.5 to 50% by mass relative to the total mass of the adhesive composition, and it is more preferable that the content of an acid addition salt of asenapine in the adhesive composition be 4.2 to 21% by mass.

It is preferable that the content of the (meth)acrylic acid ester (co)polymer in the adhesive composition be 50 to 98% by mass relative to the total mass of the adhesive composition, and it is more preferable that the content of the (meth)acrylic acid ester (co)polymer in the adhesive composition be 70 to 96% by mass. When the content of the (meth)acrylic acid ester (co)polymer is 50% by mass or more, cohesion is not decreased and the adhesion tends not to be decreased.

It is preferable that the content of the desalting agent in the adhesive composition be 0.05 to 20% by mass relative to the total mass of the adhesive composition, and it is more preferable that the content of the desalting agent in the adhesive composition be 0.1 to 10% by mass.

### EXAMPLES

The present invention will be described in more detail below with showing Examples and Comparative Examples. DURO-TAK 87-2516, DURO-TAK 87-4287, DURO-TAK 87-2194, DURO-TAK 87-900A and DURO-TAK 87-2510 were calculated based on the mass of the solid content.

(1) Manufacturing of Patches

The components described in Table 1 were each weighed out and solvents were added there as needed and the mixtures were mixed to obtain adhesive compositions. The obtained adhesive compositions were applied to polyester release liners and the solvents were dried and removed to form adhesive layers. Polyester films (backings) were laminated on the obtained adhesive layers and the obtained sheets were cut as appropriate to obtain desired patches. The figures in Table 1 refer to figures in % by mass.

TABLE 1

| | Comparative | Comparative | Comparative | Comparative |
|---|---|---|---|---|
| | Example 1 | Example 2 | Example 3 | Example 4 |
| Asenapine | 15 | 15 | 15 | 15 |
| Asenapine | — | — | — | — |
| maleate | | | | |
| DURO-TAK | 85 | 76.6 | 74.9 | 78.6 |
| 87-2516 | | | | |
| Disodium | — | 8.4 | — | — |
| maleate | | | | |
| Monosodium | — | — | 10.1 | — |
| maleate | | | | |
| trihydrate | | | | |
| Monolithium | — | — | — | 6.4 |
| maleate | | | | |
| Dipotassium | — | — | — | — |
| maleate | | | | |
| Sodium | — | — | — | — |
| hydroxide | | | | |
| Lithium | — | — | — | — |
| hydroxide | | | | |
| Total | 100 | 100 | 100 | 100 |

| | Comparative | | |
|---|---|---|---|
| | Example 5 | Example 1 | Example 2 |
| Asenapine | 15 | — | — |
| Asenapine | — | 21.1 | 21.1 |
| maleate | | | |
| DURO-TAK | 74.9 | 74.7 | 76.4 |
| 87-2516 | | | |
| Disodium | — | — | — |
| maleate | | | |
| Monosodium | — | — | — |
| maleate | | | |
| trihydrate | | | |
| Monolithium | — | — | — |
| maleate | | | |
| Dipotassium | 10.1 | — | — |
| maleate | | | |
| Sodium | — | 4.2 | — |
| hydroxide | | | |
| Lithium | — | — | 2.5 |
| hydroxide | | | |
| Total | 100 | 100 | 100 |

(2) Measurement of Amounts of Degradation Products Produced

The adhesive layers of the obtained patches were immersed in 10 mL of tetrahydrofuran (a grade for high performance liquid chromatography) and were extracted, and 40 mL of the solvent described below as a mobile phase was added there and the mixtures were filtered, and then the amounts of degradation products produced of asenapine were measured by high performance liquid chromatography under the following analysis conditions. The amounts of degradation products produced of asenapine were shown as the values of the area under the curve of the peaks corresponding to the degradation products of asenapine relative to the area under the curve of the peak corresponding to asenapine in the obtained chromatograms. The amounts of degradation products produced of asenapine were calculated taking the area under the curve of the peak corresponding to asenapine as 100.

### <Analysis Conditions>

### Column: TSK-gel ODS-80 Ts

Mobile phase: methanol: 0.1% phosphoric acid (added with 10 mM
sodium dodecyl sulfate)=75:25
Measurement wavelength: 210 nm
Flow rate: 1.0 mL/min
Column temperature: 40° C.
Sample injection volume: 10 μL
The peak having the retention time shorter than the retention time of asenapine is referred to as “Degradation product 1 of asenapine” and the peak having retention time longer than the retention time of asenapine is referred to as “Degradation product 2 of asenapine”.

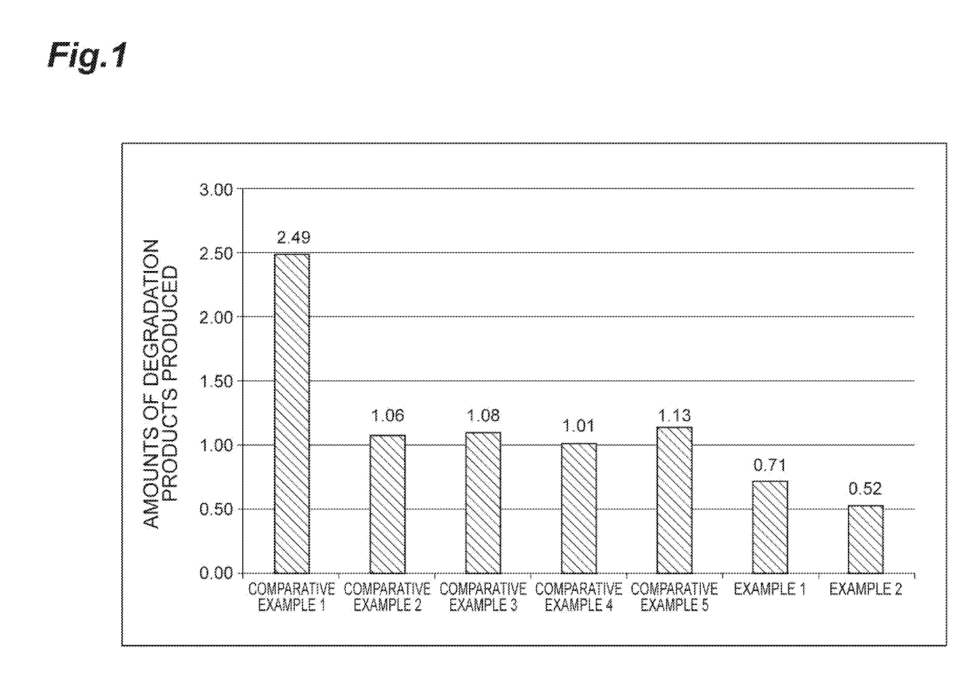

The results are shown in Table 2 and FIG. 1. The degradation products of asenapine of the patches of Examples 1 and 2 were remarkably reduced compared to those of the patches of Comparative Example 1 to 5.

TABLE 2

| | Comparative | Comparative | Comparative | Comparative |
|---|---|---|---|---|
| | Example 1 | Example 2 | Example 3 | Example 4 |
| Asenapine | 100 | 100 | 100 | 100 |
| Degradation | 2.09 | 0.65 | 0.72 | 0.65 |
| product 1 | | | | |
| Degradation | 0.40 | 0.41 | 0.36 | 0.36 |
| product 2 | | | | |
| Total of | 2.49 | 1.06 | 1.08 | 1.01 |
| Degradation | | | | |
| products | | | | |

| | Comparative | | |
|---|---|---|---|
| | Example 5 | Example 1 | Example 2 |
| Asenapine | 100 | 100 | 100 |
| Degradation | 0.78 | 0.51 | 0.34 |
| product 1 | | | |
| Degradation | 0.35 | 0.20 | 0.18 |
| product 2 | | | |
| Total of | 1.13 | 0.71 | 0.52 |
| Degradation | | | |
| products | | | |

(3) Manufacturing of Patches

The components described in Table 3 were each weighed out and solvents were added there as needed and the mixtures were mixed to obtain adhesive compositions. The obtained adhesive compositions were applied to polyester release liners, and the solvent was dried and removed to form adhesive layers having a thickness of about 100 μm. Polyester films (backings) were laminated on the obtained adhesive layers and the obtained sheets were cut as appropriate to obtain desired patches. The figures in Table 3 refer to figures in % by mass.

TABLE 3

| | Comparative | Comparative | Comparative | |
|---|---|---|---|---|
| | Example 6 | Example 7 | Example 8 | Example 3 |
| Asenapine | 15 | 15 | 15 | — |
| Asenapine | — | — | — | 21.1 |
| maleate | | | | |
| DURO-TAK | 85 | 76.6 | 74.9 | 74.7 |
| 87-900A | | | | |
| DURO-TAK | — | — | — | — |
| 87-2510 | | | | |
| Disodium | — | 8.4 | — | — |
| maleate | | | | |
| Monosodium | — | — | 10.1 | — |
| maleate | | | | |
| trihydrate | | | | |
| Sodium | — | — | — | 4.2 |
| hydroxide | | | | |
| Total | 100 | 100 | 100 | 100 |
| | Comparative | Comparative | Comparative | |
| | Example 9 | Example 10 | Example 11 | Example 4 |
| Asenapine | 15 | 15 | 15 | — |
| Asenapine | — | — | — | 21.1 |
| maleate | | | | |
| DURO-TAK | — | — | — | — |
| 87-900A | | | | |
| DURO-TAK | 85 | 76.6 | 74.9 | 74.7 |
| 87-2510 | | | | |
| Disodium | — | 8.4 | — | — |
| maleate | | | | |
| Monosodium | — | — | 10.1 | — |
| maleate | | | | |
| trihydrate | | | | |
| Sodium | — | — | — | 4.2 |
| hydroxide | | | | |
| Total | 100 | 100 | 100 | 100 |

(4) Measurement of Amounts of Degradation Products Produced

The adhesive layers of the obtained patches were immersed in 10 mL of tetrahydrofuran (a grade for high performance liquid chromatography) and were extracted, and 40 mL of the solvent described above as a mobile phase was added there and the mixtures were filtered, and then the amounts of degradation products produced of asenapine were measured by high performance liquid chromatography under the above mentioned analysis conditions.

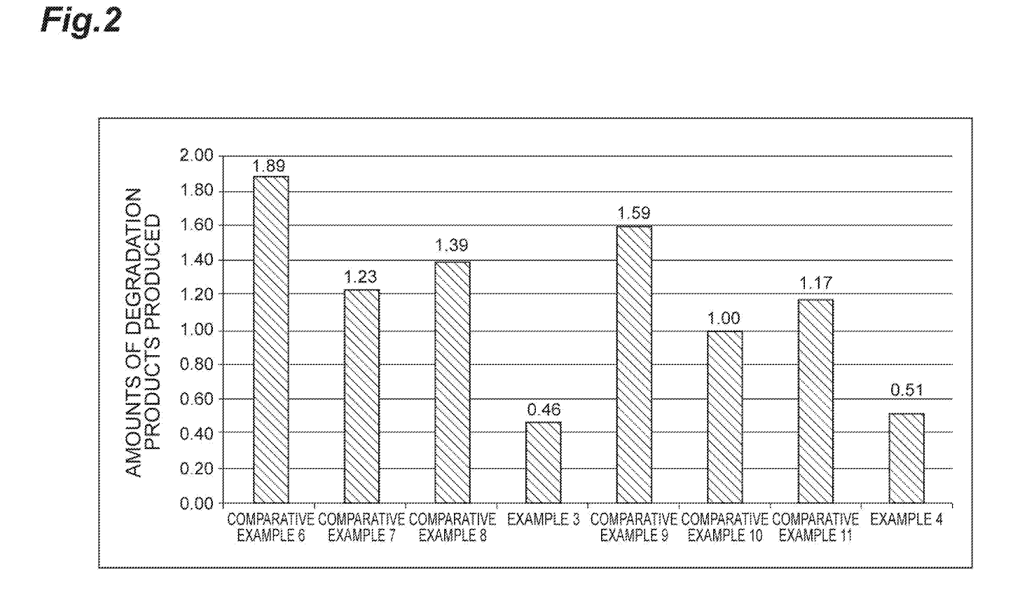

The results are shown in Table 4 and FIG. 2. The degradation products of asenapine of the patches of Examples 3 and 4 were remarkably reduced compared to those of the patches of Comparative Example 6 to 11.

TABLE 4

| | Comparative | Comparative | Comparative | |
|---|---|---|---|---|
| | Example 6 | Example 7 | Example 8 | Example 3 |
| Asenapine | 100 | 100 | 100 | 100 |
| Degradation | 1.77 | 1.14 | 1.3 | 0.43 |
| product 1 | | | | |
| Degradation | 0.12 | 0.09 | 0.09 | 0.03 |
| product 2 | | | | |
| Total of | 1.89 | 1.23 | 1.39 | 0.46 |
| Degradation | | | | |
| products | | | | |
| | Comparative | Comparative | Comparative | |
| | Example 9 | Example 10 | Example 11 | Example 4 |
| Asenapine | 100 | 100 | 100 | 100 |
| Degradation | 1.42 | 0.85 | 1.02 | 0.26 |
| product 1 | | | | |
| Degradation | 0.17 | 0.15 | 0.15 | 0.25 |
| product 2 | | | | |
| Total of | 1.59 | 1.00 | 1.17 | 0.51 |
| Degradation | | | | |
| products | | | | |

(5) Manufacturing of Patches

The components described in Table 5 were each weighed out and solvents were added there as needed and the mixtures were mixed to obtain adhesive compositions. The obtained adhesive compositions were applied to polyester release liners and the solvents were dried and removed to form adhesive layers. Polyester films (backings) were laminated on the obtained adhesive layers and the obtained sheets were cut as appropriate to obtain desired patches. The figures in Table 5 refer to figures in % by mass.

TABLE 5

| | Comparative | Comparative | Comparative | |
|---|---|---|---|---|
| | Example 12 | Example 13 | Example 14 | Example 5 |
| Asenapine | 15 | 15 | 15 | — |
| Asenapine | — | — | — | 21.1 |
| maleate | | | | |
| DURO-TAK | 85 | 76.6 | 74.9 | 74.7 |
| 87-4287 | | | | |
| DURO-TAK | — | — | — | — |
| 87-2194 | | | | |
| Disodium | — | 8.4 | — | — |
| maleate | | | | |
| Monosodium | — | — | 10.1 | — |
| maleate | | | | |
| trihydrate | | | | |
| Sodium | — | — | — | 4.2 |
| hydroxide | | | | |
| Total | 100 | 100 | 100 | 100 |
| | Comparative | Comparative | Comparative | |
| | Example 15 | Example 16 | Example 17 | Example 6 |
| Asenapine | 15 | 15 | 15 | — |
| Asenapine | — | — | — | 21.1 |
| maleate | | | | |
| DURO-TAK | — | — | — | — |
| 87-4287 | | | | |
| DURO-TAK | 85 | 76.6 | 74.9 | 74.7 |
| 87-2194 | | | | |
| Disodium | — | 8.4 | — | — |
| maleate | | | | |
| Monosodium | — | — | 10.1 | — |
| maleate | | | | |
| trihydrate | | | | |
| Sodium | — | — | — | 4.2 |
| hydroxide | | | | |
| Total | 100 | 100 | 100 | 100 |

(6) Measurement of Amounts of Degradation Products Produced

The adhesive layers of the obtained patches were immersed in 10 mL of tetrahydrofuran (a grade for high performance liquid chromatography) and were extracted, and 40 mL of the solvent described above as a mobile phase was added there and the mixtures were filtered, and then the amounts of degradation products produced of asenapine were measured by high performance liquid chromatography under the above mentioned analysis conditions.

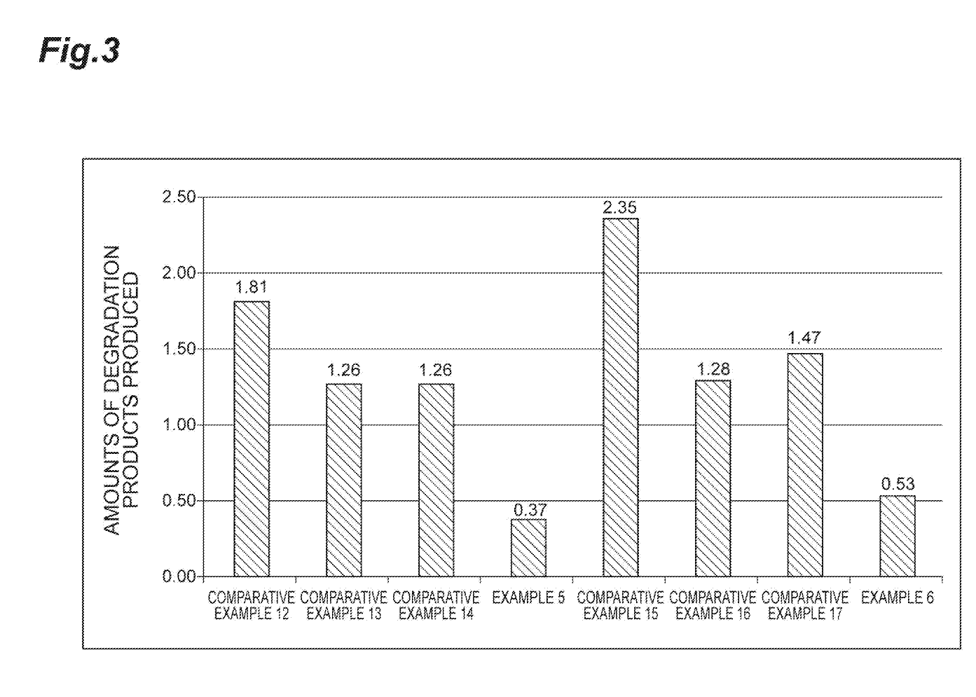

The results are shown in Table 6 and FIG. 3. The degradation products of asenapine of the patches of Examples 5 and 6 were remarkably reduced compared to those of the patches of Comparative Example 12 to 17.

TABLE 6

| | Comparative | Comparative | Comparative | |
|---|---|---|---|---|
| | Example 12 | Example 13 | Example 14 | Example 5 |
| Asenapine | 100 | 100 | 100 | 100 |
| Degradation | 1.6 | 1.21 | 1.23 | 0.34 |
| product 1 | | | | |
| Degradation | 0.21 | 0.05 | 0.03 | 0.03 |
| product 2 | | | | |
| Total of | 1.81 | 1.26 | 1.26 | 0.37 |
| Degradation | | | | |
| products | | | | |
| | Comparative | Comparative | Comparative | |
| | Example 15 | Example 16 | Example 17 | Example 6 |
| Asenapine | 100 | 100 | 100 | 100 |
| Degradation | 1.98 | 0.96 | 1.15 | 0.25 |
| product 1 | | | | |
| Degradation | 0.37 | 0.32 | 0.32 | 0.28 |
| product 2 | | | | |
| Total of | 2.35 | 1.28 | 1.47 | 0.53 |
| Degradation | | | | |
| products | | | | |

(7) Investigation of Desalting Agents

A patch (Example 7) was prepared by using an equimolar amount of potassium hydroxide instead of sodium hydroxide in the patch of Example 1. The amounts of degradation products produced of asenapine in the patch of Example 7 were measured and it was found that the amounts of degradation products produced were decreased. Further, a patch was prepared by using an equimolar amount of sodium carbonate (Example 8), by using sodium bicarbonate (Example 9), by using potassium carbonate (Example 10) and by using potassium bicarbonate (Example 11) instead of sodium hydroxide in the patch of Example 1.

(8) Manufacturing of Patches

The components described in Table 7 were each weighed out and solvents were added there as needed and the mixtures were mixed to obtain adhesive compositions. The obtained adhesive compositions were applied to polyester release liners and the solvents were dried and removed to form adhesive layers having a thickness of about 100 μm. Polyester films (backings) were laminated on the obtained adhesive layers and the obtained sheets were cut as appropriate to obtain desired patches. The figures in Table 7 refer to figures in % by mass.

TABLE 7

| | Example | Example | Example | Comparative |
|---|---|---|---|---|
| | 12 | 13 | 14 | Example 18 |
| Asenapine | — | — | — | 8.5 |
| Asenapine maleate | 12 | 12 | 12 | — |
| DURO-TAK 87-2516 | 79.09 | 82.98 | 68.86 | 91.5 |
| Triethanolamine | 8.91 | — | — | — |
| Sodium bicarbonate | — | 5.02 | — | — |
| Sodium oleate | — | — | 19.14 | — |
| Total | 100 | 100 | 100 | 100 |

(9) Measurement of Amounts of Degradation Products Produced

The results are shown in Table 8. The degradation products of asenapine of the patches of Examples 12 to 14 were remarkably reduced compared to those of the patch of Comparative Example 18. “N.D.” in Table 8 means that the value is below the detection limit.

TABLE 8

| | Example | Example | Example | Comparative |
|---|---|---|---|---|
| | 12 | 13 | 14 | Example 18 |
| Degradation product 1 | N.D. | N.D. | 0.16 | 1.22 |
| Degradation product 2 | 0.10 | 0.12 | 0.10 | 0.31 |
| Total of Degradation | 0.10 | 0.12 | 0.26 | 1.53 |
| products | | | | |

(10) Manufacturing of Patches

The components described in Table 9 were each weighed out and solvents were added there as needed and the mixtures were mixed to obtain adhesive compositions. The obtained adhesive compositions were applied to polyester release liners and the solvents were dried and removed to form adhesive layers having a thickness of about 100 μm. Polyester films (backings) were laminated on the obtained adhesive layers and the obtained sheets were cut as appropriate to obtain desired patches. The figures in Table 9 refer to figures in % by mass.

TABLE 9

| | Example 15 | Example 16 | Example 17 |
|---|---|---|---|
| Asenapine maleate | 0.50 | 4.22 | 8.44 |
| DURO-TAK 87-2516 | 99.40 | 94.94 | 89.88 |
| Sodium hydroxide | 0.10 | 0.84 | 1.68 |
| Total | 100 | 100 | 100 |
| | Example 18 | Example 19 | Example 20 |
| Asenapine maleate | 21 | 35 | 50 |
| DURO-TAK 87-2516 | 74.82 | 58.03 | 40.05 |
| Sodium hydroxide | 4.18 | 6.97 | 9.95 |
| Total | 100 | 100 | 100 |

(11) Measurement of Amounts of Degradation Products Produced

The result is shown in Table 10. The degradation products of asenapine of the patches of Examples 15 to 20 were remarkably small.

TABLE 10

| | Example 15 | Example 16 | Example 17 |
|---|---|---|---|
| Degradation product 1 | 0.34 | 0.27 | 0.45 |
| Degradation product 2 | 0.08 | 0.23 | 0.24 |
| Total of Degradation | 0.42 | 0.50 | 0.69 |
| products | | | |
| | Example 18 | Example 19 | Example 20 |
| Degradation product 1 | 0.51 | 0.35 | 0.06 |
| Degradation product 2 | 0.20 | 0.12 | 0.00 |
| Total of Degradation | 0.71 | 0.47 | 0.06 |
| products | | | |

1. A method for manufacturing a patch comprising a backing and an adhesive layer laminated on the backing, comprising:
a step of mixing an acid addition salt of asenapine, a (meth)acrylic acid ester (co)polymer and a desalting agent to obtain an adhesive composition; and
a step of shaping the adhesive composition to obtain the adhesive layer.

2. The method according to claim 1, wherein the acid in the acid addition salt of asenapine is selected from the group consisting of hydrochloric acid, hydrobromic acid, hydriodic acid, phosphoric acid, acetic acid, propionic acid, glycolic acid, maleic acid, malonic acid, succinic acid, tartaric acid, citric acid, ascorbic acid, salicylic acid and benzoic acid.

3. The method according to claim 1, wherein the desalting agent is selected from the group consisting of an alkali metal hydroxide, an alkali metal salt, an alkaline-earth metal hydroxide, an alkaline-earth metal salt and a low molecular weight amine.

4. A method for stabilizing asenapine in an adhesive composition comprising a (meth)acrylic acid ester (co)polymer, comprising:
a step of mixing an acid addition salt of asenapine, a (meth)acrylic acid ester (co)polymer and a desalting agent to obtain the adhesive composition.

5. A method for inhibiting the degradation of asenapine in a manufacturing process of a patch comprising an adhesive layer comprising asenapine, comprising:
a step of mixing an acid addition salt of asenapine, a (meth)acrylic acid ester (co)polymer and a desalting agent to obtain an adhesive composition; and
a step of shaping the adhesive composition to obtain the adhesive layer.

6. The method according to claim 2, wherein the desalting agent is selected from the group consisting of an alkali metal hydroxide, an alkali metal salt, an alkaline-earth metal hydroxide, an alkaline-earth metal salt and a low molecular weight amine.
